(19) United States
(12) Patent Application Publication
(10) Pub. No.: US 2022/0096460 A1
(43) Pub. Date: Mar. 31, 2022

(54) ABUSE-RESISTANT LONG-ACTING RELEASE OPIOID PRODRUGS

(71) Applicant: SUZHOU RUNXINDATAI PHARMACEUTICS LTD CO., Buzhou, Jiangsu Province (CN)

(72) Inventor: Hui OUYANG, Irvine, CA (US)

(21) Appl. No.: 17/423,339

(22) PCT Filed: Jan. 16, 2020
(86) PCT No.: PCT/US2020/013782

§ 371 Date: Jul. 15, 2021

Related U.S. Application Data
(60) Provisional application No. 62/793,684, filed on Jan. 17, 2019.

(51) Int. Cl. A61K 31/485; C07D 215/14; C07D 491/08; A61K 47/14

(52) U.S. Cl. CPC A61K 31/485; A61K 47/14; C07D 491/08; C07D 215/14

(57) ABSTRACT

There are provided, prodrugs of opioid such as levorphanol or morphine, having enhanced physical and chemical stability to resist tampering and to make long- acting release formulations, and pharmaceutically accepted salts and solvates thereof. There are also provided methods of using the disclosed compounds as abuse deterrent products.

### CROSS REFERENCE TO RELATED APPLICATIONS

This application claims priority of U.S. Provisional Application No. 62/793,684, filed on Jan. 17, 2019, the content of which is incorporated herein by reference in its entirety.

### FIELD OF INVENTION

In various embodiments, the present invention relates generally to abuse-resistant products to combat prescription opioid drug abuse. For example, in some embodiments, the present invention relates to the preparation and application of abuse-resistant long-acting release opioid products.

### BACKGROUND OF THE INVENTION

Prescription opioid drugs including morphine and levorphanol are widely used, e.g., for the treatment of pain. The abuse of prescription opioid drugs is ‘epidemic’ in the United States, which is associated with heavy social and economic costs. Despite the heavy social and economic costs associated with the abuse of prescription opioid drugs, opioids are essential for improving the care and outcomes for the 100 million adults living with chronic pain in the United States.

There are numerous technological means to battle prescription opioid abuse. One way is to employ so-called ‘abuse-deterrent’ formulation that resists the tampering or extraction of opioids from the oral tablets for snorting or injection. However, abusers can always find a way to go around the ‘abuse-deterrent’ formulation. Another way is to add deterrent chemical, such as naloxone or naltrexone, to opioid formulation, which is unsuccessful in reducing opioid abuse potential.

Formulation technology has led to limited success in developing abuse-deterrent opioid products. Alternative methods for combating opioid abuse are needed.

### BRIEF SUMMARY OF THE INVENTION

In various embodiments, the present invention provides novel prodrugs of opioids, such as levorphanol and morphine. In some embodiments, the prodrugs herein can have physical, chemical and biological activities that resist/deter drug tampering and/or achieve long-acting release profile in vivo. The long-acting release profile makes it possible for doctors to use the opioid products only in hospital or doctor's office, limiting patients' access of the opioids outside the clinics and avoiding the potential of opioid abuse altogether.

In some specific embodiments, the novel prodrug is a prodrug of levorphanol. In some embodiments, the prodrug is a compound of Formula 1, or a pharmaceutically acceptable salt thereof:

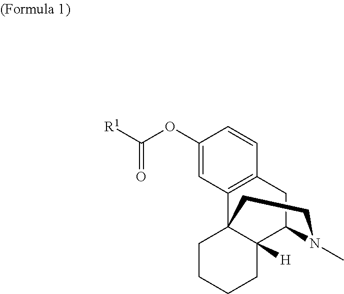

wherein R¹ is defined herein. In some embodiments, R¹ is an unsubstituted straight alkyl chain having 7-30 carbons. In some embodiments, R¹ is an unsubstituted branched alkyl chain having 7-30 carbons. In some embodiments, R¹ is a substituted or unsubstituted straight or branched alkyl chain having a total number of carbons of 7-30. In some embodiments, R¹ is an unsubstituted straight alkyl chain having a formula of CH₃(CH₂)ₙ—, wherein n is an integer of 8-24 (e.g., 10-24). In some specific embodiments, R¹ is selected from CH₃(CH₂)₁₀—, CH₃(CH₂)₁₂—, CH₃(CH₂)₁₄—, CH₃(CH₂)₁₆—, and CH₃(CH₂)₁₈—.

In some embodiments, the novel prodrug is a prodrug of morphine. In some embodiments, the prodrug is of Formula 2 (2A, 2B, or 2C), or a pharmaceutically acceptable salt thereof:

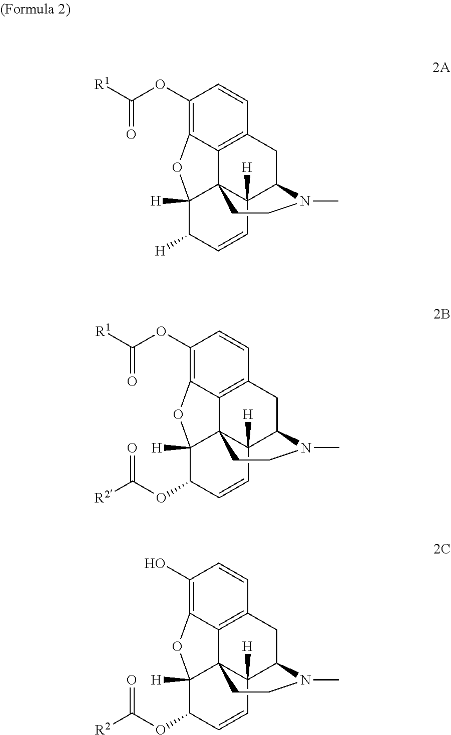

wherein R² and R²′ are defined herein. In some embodiments, the prodrug can be characterized as having a formula 2A. In some embodiments, the prodrug can be characterized as having a formula 2B. In some embodiments, the prodrug can be characterized as having a formula 2C. In some embodiments, R² and R²′ are independently an unsubstituted straight alkyl chain having 7-30 carbons. In some embodiments, R² and R²′ are independently an unsubstituted branched alkyl chain having 7-30 carbons. In some embodiments, R² and R²′ are independently a substituted or unsubstituted straight or branched alkyl chain having a total number of carbons of 7-30. In some embodiments, R² and R²′ are independently an unsubstituted straight alkyl chain having a formula of CH₃(CH₂)ₙ—, wherein n is an integer of 8-24 (e.g., 10-24). In some specific embodiments, R² and R²′ are independently selected from CH₃(CH₂)₁₀—, CH₃(CH₂)₁₂—, CH₃(CH₂)₁₄—, CH₃(CH₂)₁₆—, and CH₃(CH₂)₁₈—. R² and R²′ can be the same or different from each other.

In some embodiments, the present disclosure provides a compound selected from Compound Nos. 1-8, or a pharmaceutically acceptable salt thereof.

Certain embodiments of the present disclosure are directed to a pharmaceutical composition comprising the prodrugs herein. In some embodiments, the pharmaceutical composition is an abuse-deterrent formulation. In some embodiments, the pharmaceutical composition can comprise a compound of Formula 1 or 2 (2A, 2B, or 2C), a long-chain fatty acid ester of levorphanol, a long-chain fatty acid ester of morphine, any of the Compound Nos. 1-8, or a pharmaceutically acceptable salt thereof. In some embodiments, the pharmaceutical composition can be formulated for injection, such as subcutaneous or intramuscular injection. In some embodiments, the pharmaceutical composition can be resistant towards (e.g., substantially stable under) common tampering conditions, such as baking soda or vinegar mediated hydrolysis at pH of about 8.3 or 2.4, respectively, or citric acid mediated hydrolysis at a pH of about 1.6. In some embodiments, the pharmaceutical composition comprising the compound of Formula 1 or 2 (2A, 2B, or 2C) or pharmaceutically acceptable salt thereof can provide a long acting release of levorphanol or morphine in a subject user. In some embodiments, the pharmaceutical composition can, after administration, release levorphanol and morphine, or a metabolite thereof, in a subject user over an extended period of time, such as at least 1 day, at least 2 days, or at least 3 days.

Certain embodiments of the present disclosure are also directed to methods of treating pain (e.g., chronic pain). In some embodiments, the method comprises administering to a subject in need thereof a therapeutically effective amount of the prodrug of the present disclosure, or a pharmaceutical composition (e.g., an injectable formulation) comprising the prodrug. In some embodiments, the method is for treating neuropathic pain in a subject in need thereof, the method comprising administering to the subject a therapeutically effective amount of the compound of Formula 1, a long-chain fatty acid ester of levorphanol, or pharmaceutically acceptable salt thereof, or a pharmaceutical composition comprising the compound of Formula 1, long-chain fatty acid ester of levorphanol, or pharmaceutically acceptable salt thereof. In some embodiments, the administering can be an injection, such as a subcutaneous or intramuscular injection. In some embodiments, the prodrug is a compound of Formula 1 or 2 (2A, 2B, or 2C), or a pharmaceutically acceptable salt thereof. In some embodiments, the prodrug can be a compound of Formula 1 or 2 (e.g., any of the Compound Nos. 1-8), or a pharmaceutically acceptable salt thereof.

In some embodiments, the present disclosure also provides methods of reducing a likelihood of abuse of a controlled substance. In some embodiments, the method comprises providing a prodrug of the present disclosure, and formulating the prodrug in an abuse-deterrent formulation. In some embodiments, the abuse-deterrent formulation is an injectable formulation, such as a subcutaneous or intramuscular injectable formulation. In some embodiments, the abuse-deterrent formulation is resistant towards (e.g., substantially stable under) common tampering conditions, such as baking soda or vinegar mediated hydrolysis at pH of about 8.3 or 2.4, respectively, or citric acid mediated hydrolysis at a pH of about 1.6. In some embodiments, the abuse-deterrent formulation comprises micelles comprising the prodrug. In some embodiments, the method further comprising restricting the administration of the abuse-deterrent formulation to a hospital setting. In some embodiments, the abuse-deterrent formulation also provides a long acting release of levorphanol or morphine in a subject user. For example, the abuse-deterrent formulation can, after administration, release levorphanol or morphine, or a metabolite thereof, in a subject user over an extended period of time, such as at least 1 day, at least 2 days, or at least 3 days. In some embodiments, the prodrug is a compound of Formula 1 or 2 (2A, 2B, or 2C), a long-chain fatty acid ester of levorphanol, a long-chain fatty acid ester of morphine, or a pharmaceutically acceptable salt thereof. In some embodiments, the prodrug can be a compound of Formula 1 or 2 (e.g., any of the Compound Nos. 1-8), or a pharmaceutically acceptable salt thereof.

### DETAILED DESCRIPTION OF THE INVENTION

In various embodiments, the present disclosure relates to prodrugs of exemplary opioids, in particular, prodrugs of levorphanol and morphine, pharmaceutical compositions comprising the prodrugs, methods of preparing the prodrugs, and methods of use the prodrugs, for example, in treating pain (e.g., neuropathic pain or chronic pain) and/or in reducing drug abuse. The present disclosure is in part based on the discovery that certain prodrugs of opioids, such as a long-chain fatty acid ester prodrug, can have physical, chemical and biological activities that resist/deter drug tampering and/or achieve long-acting release profile in vivo. The long-acting release profile (of the opioid) makes it possible for doctors to use the prodrug only in hospital or doctor's office, limiting patients' access of the opioids outside the clinics and avoiding the potential of opioid abuse altogether. This general prodrug technology to combat prescription opioids abuse was also described in our previous patent application, PCT/US2018/042880, filed Jul. 19, 2018, the content of which is herein incorporated by reference in its entirety. As described in PCT/US2018/042880, representative prodrugs of two opioids, hydromorphone and oxymorphone, can be stable towards common tampering conditions and can be used to achieve a long-acting release profile in vivo. Thus, PCT/US2018/042880 shows that the general prodrug approach disclosed therein can be used to control prescription opioids abuse.

The prodrugs of the present disclosure are useful for treating any of the diseases or disorders where administering the parent drugs (e.g., levorphanol or morphine) are useful. For example, both levorphanol and morphine are known to be useful for treating pain. Thus, the prodrugs herein can be useful for treating pain, including moderate to severe pain and chronic pain, etc. Further, levorphanol has been proposed as a good substitute of methadone. See e.g., Frommer, E. Palliative care: Research and Treatment 8:7-10 (2014); see also Support Care Cancer 15:259-264 (2007). Methadone has been a stalwart pharmacologic option for the management of opioid drug dependence for many years. It substitutes for opioid agonists and possesses certain pharmacokinetic properties that confer characteristics preferable to those of other opioids. Unfortunately, methadone has some undesirable, and even worrisome, features, including issues of safety, such as causing QT prolongation. Unlike methadone, levorphanol is a more potent NMDA antagonist, therefore can be useful for treating neuropathic pain. Levorphanol has a shorter plasma half-life yet longer duration of action, has no CYP450 interaction or QT prolongation risk, can be a viable option in the elderly, palliative care, and requires little to no need for co-administration of adjuvant analgesics. Thus, prodrugs of levorphanol of the present disclosure can also be used as a substitute of methadone, for example, for treating neuropathic pain.

Morphine is naturally found within the opium poppy plant. Among the natural opioids found within the poppy, morphine is the most abundant and potent, making it a popular pain reliever since its discovery. It is used to relieve moderate to severe pain, usually after surgery or traumatic injury. Despite the side effects of Morphine, its effectiveness as a pain reliever has kept it a staple for patients who suffer from chronic pain. It is the golden standard of pain killer. The prodrugs of morphine of the present disclosure can also be used similarly, for example, for treating moderate to severe pain and for treating chronic pain.

### I. DEFINITIONS

The abbreviations used herein have their conventional meaning within the chemical and biological arts.

Where moieties are specified by their conventional chemical formulae, written from left to right, they equally encompass the chemically identical moieties that would result from writing the structure from right to left, e.g., —CH₂O— is equivalent to —OCH₂—.

It is also meant to be understood that a specific embodiment of a variable moiety herein may be the same or different as another specific embodiment having the same identifier.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

As used herein, the term “alkyl” as used by itself or as part of another group refers to a straight- or branched-chain saturated aliphatic hydrocarbon. In some embodiments, the alkyl can include one to thirty carbon atoms (i.e., C₁₋₃₀ alkyl or alternatively expressed as C₁-C₃₀ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. Exemplary C₁₋₄ alkyl groups include methyl, ethyl, propyl, isopropyl, butyl, sec-butyl, tert-butyl, and iso-butyl.

As used herein, the term “cycloalkyl” as used by itself or as part of another group refers to saturated and partially unsaturated (e.g., containing one or two double bonds) cyclic aliphatic hydrocarbons containing one to three rings having from three to twelve carbon atoms (i.e., C₃₋₁₂ cycloalkyl) or the number of carbons designated. In one embodiment, the cycloalkyl group has two rings. In one embodiment, the cycloalkyl group has one ring. In another embodiment, the cycloalkyl group is a C₃₋₈ cycloalkyl group. In another embodiment, the cycloalkyl group is a C₃₋₆ cycloalkyl group. “Cycloalkyl” also includes ring systems wherein the cycloalkyl ring, as defined above, is fused with one or more aryl or heteroaryl groups wherein the point of attachment is on the cycloalkyl ring, and in such instances, the number of carbons continue to designate the number of carbons in the cycloalkyl ring system. Non-limiting exemplary cycloalkyl groups include cyclopropyl, cyclobutyl, cyclopentyl, cyclohexyl, cycloheptyl, cyclooctyl, norbornyl, decalin, adamantyl, cyclopentenyl, and cyclohexenyl.

As used herein, the term “alkenyl” as used by itself or as part of another group refers to a straight- or branched-chain aliphatic hydrocarbon containing one or more (e.g., 1, 2, or 3) carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more (e.g., 1, 2, or 3) carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

The term “substituted alkyl” means an alkyl group with carbon at any position connecting to a functional group. The functional group can be, but not limited to, alkoxy, alkylamino, alkylthio, heteroalkyl, cycloalkyl, heterocycloalkyl, halo, aryl, heteroalkyl, heteroatom ring heteroatom, or heteroaryl group.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

As used herein, the term “alkanoyl” as used by itself or as part of another group refers to —C(O)R^{a1}, wherein R^{a1} is hydrogen or an alkyl. For example, C₁ alkanoyl refers to —C(O)H, C₂ alkanoyl refers to —C(O)CH_{3.}

The term “heteroalkyl,” by itself or in combination with another term, means, unless otherwise stated, a stable straight or branched chain, consisting of the stated number of carbon atoms and a heteroatom selected from the group consisting of O, N, P, Si and S, and wherein the nitrogen and sulfur atoms may optionally be oxidized and the nitrogen heteroatom may optionally be quaternized. Typically, the heteroalkyl is a saturated chain. However, for brevity, the heteroalkyl herein also includes unsaturated chain, such as those containing one or more carbon-carbon double bonds. The heteroatom(s) O, N, P and S and Si may be placed at any interior position of the heteroalkyl group or at the position at which the heteroalkyl group is attached to the remainder of the molecule. Examples of heteroalkyls include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. As described above, heteroalkyl groups, as used herein, include those groups that are attached to the remainder of the molecule through a carbonyl or heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “halo” or “halogen,” by themselves or as part of another substituent, mean, unless otherwise stated, a fluorine, chlorine, bromine, or iodine atom.

As used herein, the term “heterocycle” or “heterocyclyl” as used by itself or as part of another group refers to saturated and partially unsaturated (e.g., containing one or two double bonds) cyclic groups containing one, two, or three rings having from three to fourteen ring members (i.e., a 3- to 14-membered heterocycle) and at least one heteroatom. Each heteroatom can be independently selected, e.g., from the group consisting of oxygen, sulfur, including sulfoxide and sulfone, and/or nitrogen atoms, which can be quaternized. The term “heterocyclyl” is meant to include cyclic ureido groups such as imidazolidinyl-2-one, cyclic amide groups such as β-lactam, γ-lactam, δ-lactam and ε-lactam, and cyclic carbamate groups such as oxazolidinyl-2-one. In one embodiment, the heterocyclyl group is a 4-, 5-, 6-, 7- or 8-membered cyclic group containing one ring and one or two oxygen and/or nitrogen atoms. The heterocyclyl can be optionally linked to the rest of the molecule through a carbon or nitrogen atom. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more cycloalkyl groups wherein the point of attachment is either on the cycloalkyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

As used herein, the term “aryl” as used by itself or as part of another group refers to a monocyclic, bicyclic or tricyclic aromatic ring system having from six to fourteen carbon atoms (i.e., C₆₋₁₄ aryl) and zero heteroatoms. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). “Aryl” also includes ring systems wherein the aryl ring, as defined above, is fused with one or more cycloalkyl or heterocyclyl groups wherein the radical or point of attachment is on the aryl ring, and in such instances, the number of carbon atoms continue to designate the number of carbon atoms in the aryl ring system.

As used herein, the term “heteroaryl” or “heteroaromatic” refers to monocyclic, bicyclic or tricyclic aromatic ring systems having 5 to 14 ring atoms (i.e., a 5- to 14-membered heteroaryl) and 1, 2, 3, or 4 heteroatoms independently chosen from oxygen, nitrogen and sulfur. In one embodiment, the heteroaryl has one heteroatom, e.g., one nitrogen. In another embodiment, the heteroaryl has 6 ring atoms, e.g., pyridyl. In one embodiment, the heteroaryl is a bicyclic heteroaryl having 8 to 10 ring atoms, e.g., a bicyclic heteroaryl having 1, 2, or 3 nitrogen ring atoms, such as quinolyl. As used herein, the term “heteroaryl” is also meant to include possible N-oxides. “Heteroaryl” includes ring systems wherein the heteroaryl ring, as defined above, is fused with one or more cycloalkyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system.

For brevity, the term “aryl” when used in combination with other terms (e.g., aryloxy, arylthioxy, arylalkyl) includes both aryl and heteroaryl rings as defined above. The term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

An “optionally substituted” group, such as an optionally substituted alkyl, an optionally substituted alkenyl, an optionally substituted alkynyl, an optionally substituted cycloalkyl, an optionally substituted heterocyclyl, an optionally substituted aryl, and an optionally substituted heteroaryl groups, refers to the respective group that is unsubstituted or substituted. In general, the term “substituted”, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Typically, a “stable” compound is a compound that can be prepared and isolated and whose structure and properties remain or can be caused to remain essentially unchanged for a period of time sufficient to allow use of the compound for the purposes described herein (e.g., therapeutic administration to a subject). Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle.

Preferred substituent moieties for each type of radical are provided below.

Substituent moieties for the alkyl, alkenyl, alkynyl, cycloalkyl, heterocyclyl, and heteroalkyl radicals can be one or more of a variety of groups selected from, but not limited to: deuterium, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl.

Similar to the substituent moieties described for the alkyl radical, substituent moieties for the aryl and heteroaryl groups are varied and may be selected from, for example: deuterium, halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituent moieties on adjacent atoms of the aryl or heteroaryl ring may optionally form a ring of the formula -Q′—C(O)-(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(P)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

The term “pharmaceutically acceptable salts” is meant to include salts of the compounds which are prepared with relatively nontoxic acids or bases, depending on the particular substituent moieties found on the compounds described herein. When prodrugs of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When prodrugs of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., J. Pharmaceutical Sciences, 66:1-19 (1977)). Certain specific prodrugs of the present disclosure contain both basic and acidic functionalities that allow the compounds to be converted into either base or acid addition salts.

The neutral forms of the compounds can be preferably regenerated by contacting the salt with a base or acid and isolating the parent compound in the conventional manner when desired. The parent form of the compound differs from the various salt forms in certain physical properties, such as solubility in polar solvents.

As used herein, the term “prodrug(s) of the present disclosure” refers to any of the compounds described herein according to Formula 1, or 2 (including 2A, 2B, and 2C), a long-chain fatty acid ester prodrug of levorphanol, a long-chain fatty acid ester prodrug of morphine, or any of Compound Nos. 1-8, isotopically labeled compound(s) thereof (e.g., deuterium enriched compounds), possible stereoisomers thereof (including diastereoisomers, enantiomers, and racemic mixtures), tautomers thereof, conformational isomers thereof, and/or pharmaceutically acceptable salts thereof (e.g., acid addition salt such as HCl salt or base addition salt such as Na salt). Hydrates and solvates of the prodrugs of the present disclosure are considered compositions of the present disclosure, wherein the prodrug(s) is in association with water or solvent, respectively. Some of the prodrugs of the present disclosure can also exist in various polymorphic forms or amorphous forms. The prodrugs described herein include those compounds that readily undergo chemical changes under physiological conditions to provide active opioids. Additionally, prodrugs can be converted by chemical or biochemical methods in an ex vivo environment. For example, prodrugs can be slowly converted to the active compounds when placed in a transdermal patch reservoir with a suitable enzyme or chemical reagent.

Certain prodrugs of the present disclosure possess asymmetric carbon atoms (optical centers) or double bonds; the racemates, diastereomers, tautomers, geometric isomers and individual isomers are encompassed within the scope of the present invention. The prodrugs of the present disclosure do not include those which are known in the art to be too unstable to synthesize and/or isolate.

The prodrugs of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

Solid and dashed wedge bonds indicate stereochemistry as customary in the art.

The term “subject” (alternatively referred to herein as “patient”) as used herein, refers to an animal, preferably a mammal, most preferably a human, who has been the object of treatment, observation or experiment. In some embodiments, the subject can be a vertebrate such as a dog, a cat, a horse or a monkey.

### II. COMPOUNDS

In various embodiments, the present disclosure provides novel prodrugs of an opioid, pharmaceutical composition comprising the prodrug(s), and methods of preparing an abuse deterrent formulation comprising the prodrug(s), and methods of using the prodrug(s) or any of the pharmaceutical compositions, abuse deterrent formulations, for example for the treatment of pain, such as neuropathic pain and/or chronic pain. Typically, the prodrugs can be a long-chain fatty acid ester of an opioid such as levorphanol, morphine, oxymorphone, or hydromorphone, etc. Representative prodrugs of oxymorphone and hydromorphone are described in PCT/US2018/042880. The present disclosure describes prodrugs of levorphanol and morphine in more details.

In some specific embodiments, the present disclosure provides a prodrug of levorphanol.

In some embodiments, the prodrug of levorphanol is a long-chain fatty acid ester of levorphanol. A long chain fatty acid as used herein refers to an aliphatic chain fatty acid with 13 or more carbons, for example, a naturally occuring aliphatic chain fatty acid with 13-28 carbons, which can a saturated, monounsaturated (containing one carbon-carbon double bond), or polyunsaturated (containing more than one carbon-carbon double bonds) fatty acid, an example of naturally occuring long chain fatty acid is palmitic acid which has 16 carbons. Naturally occuring fatty acids herein include those fatty acids that exist in nature predominantly in the form of an ester, such as triglycerides, etc. In some embodiments, the prodrug is a compound of Formula 1, or a pharmaceutically acceptable salt thereof

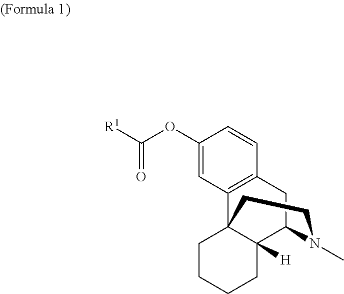

wherein R¹ is R¹⁰, —OR¹⁰, or —NHR¹⁰, wherein R¹⁰ is an optionally substituted straight or branched alkyl, alkenyl, or alkynyl chain having a total of 7-30 carbons. As used herein, the total number of carbons should be understood as including branched carbons and those from optional substituents. R¹⁰ at each occurrence is independently selected. In some embodiments, the alkyl, alkenyl, or alkynyl chain is optionally substituted with one or more hydrophobic groups. As used herein, the term “hydrophobic group” generically refers to halogen or a carbon-containing group with 2 or less heteroatoms selected from oxygen and nitrogen atoms, which typically includes no OH or NH group and no basic nitrogen atom(s). Examples of hydrophobic groups include halogen, alkyl, alkoxy, alkenyl, alkynyl, cycloalkyl, cycloalkoxy, aryls, non-basic heterocycles and heteroaryls, etc. In some embodiments, the alkyl, alkenyl, or alkynyl chain is optionally substituted with one or more (e.g., 1, 2, 3, 4, 5, or 6) groups independently selected from halogen, C₁₋₄ alkyl, C₂₋₄ alkenyl, C₂₋₄ alkynyl, C₁₋₄ alkoxy, C₃₋₆ cycloalkyl, and C₃₋₆ cycloalkoxy. In some embodiments, the alkyl, alkenyl, or alkynyl chain can be optionally substituted one or more (e.g., 1, 2, 3, 4, 5, or 6) groups independently selected from halogen, optionally substituted alkyl (e.g., C₁₋₆ alkyl), optionally substituted heteroalkyl (e.g., C₁₋₆ heteroalkyl, e.g., with 1 or 2 heteroatoms independently selected from oxygen and nitrogen), optionally substituted alkenyl (e.g., C₂₋₆ alkenyl), optionally substituted alkynyl (e.g., C₂₋₆ alkynyl), optionally substituted cycloalkyl (e.g., C₃₋₆ cycloalkyl), optionally substituted aryl (e.g., C₆₋₁₄ aryl), optionally substituted heterocycloalkyl (e.g., 5-8 membered heterocycloalkyl), optionally substituted heteroaryl (e.g., 5-10 membered heteroaryl), short peptides (e.g. mono, di, tri, or tetra-peptides), —NR¹⁰⁰R¹⁰¹, —C(═O)NR¹⁰⁰R¹⁰¹, —COOR¹⁰², and —OR¹⁰², wherein R¹⁰⁰, R¹⁰¹, and R¹⁰² are each independently hydrogen, optionally substituted alkyl (e.g., C₁₋₆ alkyl), optionally substituted heteroalkyl (e.g., C₁₋₆ heteroalkyl, e.g., with 1 or 2 heteroatoms independently selected from oxygen and nitrogen), optionally substituted cycloalkyl (e.g., C₃₋₆ cycloalkyl), optionally substituted aryl (e.g., C₆₋₁₄ aryl), optionally substituted heterocycloalkyl (e.g., 5-8 membered heterocycloalkyl), optionally substituted heteroaryl (e.g., 5-10 membered heteroaryl), wherein each of the optionally substituted groups is independently optionally substituted with one or more (e.g., 1-3) substituents selected from oxo, halogen, hydroxyl, NH₂, —NH(C₁₋₄ alkyl), —N(C₁₋₄alkyl)(C₁₋₄ alkyl), C₁₋₄ alkyl optionally substituted with 1-3 fluorine, C₁₋₄ alkoxy optionally substituted with 1-3 fluorine, C₂₋₄ alkenyl, C₂₋₄ alkynyl, C₃₋₆ cycloalkyl optionally substituted with 1-3 fluorine or 1-2 C₁₋₄ alkyl, and C₃₋₆ cycloalkoxy optionally substituted with 1-3 fluorine or 1-2 C₁₋₄ alkyl. In some embodiments, the short peptides can be a mono-, di-, tri-, or tetra-peptide, which can be derived from alpha-amino acids (e.g., D or L-amino acids) selected from alanine, isoleucine, leucine, methionine, valine, phenylalanine, tryptophan, tyrosine, asparagine, cysteine, glutamine, serine, threonine, aspartic acid, glutamic acid, arginine, histidine, lysine, glycine, and proline. As used herein, the short peptides as substituents can bond to the group which is substituted, e.g., the alkyl, alkenyl, or alkynyl chain, either through the N-terminal (e.g., through NH₂, optionally with one of the hydrogens substituted with C₁₋₄ alkyl or C₁₋₆ alkanoyl) or through the C-terminal (e.g., through —C(═O), —OC(═O), —NC(═O), etc.), with the non-connecting terminal being NH₂, or a protected derivative thereof such as N-Pg (e.g., NHC(═O)CH₃), in the case of N-terminal, or CO₂H, or ester (e.g., C₁₋₄ alkyl ester) or amide derivative thereof, in the case of C-terminal. In some embodiments, the short peptides as substituents can bond to the group which is substituted, e.g., the alkyl, alkenyl, or alkynyl chain, through the N-terminal. In some embodiments, the short peptides as substituents can bond to the group which is substituted, e.g., the alkyl, alkenyl, or alkynyl chain, through the C-terminal. In some embodiments, R¹⁰ is an unsubstituted straight alkyl chain having 7-30 (e.g., 10-24) carbons. In some embodiments, R¹⁰ is an unsubstituted branched alkyl chain having 7-30 (e.g., 10-24) carbons. In some embodiments, R¹ is an unsubstituted straight alkyl chain having 7-30 (e.g., 10-24) carbons. In some embodiments, R¹ is an unsubstituted branched alkyl chain having 7-30 (e.g., 10-24) carbons. In some embodiments, R¹ is an unsubstituted straight alkyl chain having a formula of CH₃(CH₂)ₙ—, wherein n is an integer of 8-24 (e.g., 10-24). In some specific embodiments, R¹ is selected from CH₃(CH₂)₁₀—, CH₃(CH₂)₁₂—, CH₃(CH₂)₁₄—, CH₃(CH₂)₁₆—, and CH₃(CH₂)₁₈—.

In some embodiments, the present disclosure also provides a prodrug of morphine. In some embodiments, the prodrug of morphine is a long-chain fatty acid ester of morphine, which can be a mono-ester or a di-ester. In some embodiments, the prodrug is a compound of Formula 2 (2A, 2B, or 2C), or a pharmaceutically acceptable salt thereof

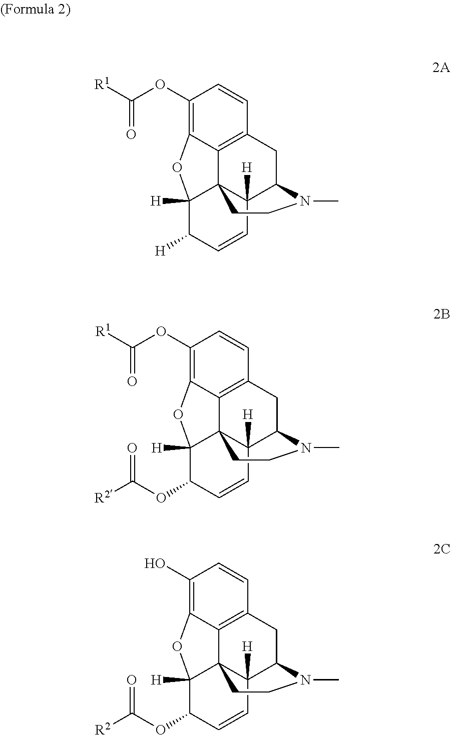

wherein R² and R²′ is R²⁰, —OR²⁰, or —NHR²⁰, wherein R²⁰ is an optionally substituted straight or branched alkyl, alkenyl, or alkynyl chain having a total number of 7-30 carbons. In some embodiments, the alkyl, alkenyl, or alkynyl chain is optionally substituted with one or more hydrophobic groups (e.g., as described herein). In some embodiments, the alkyl, alkenyl, or alkynyl chain is optionally substituted with one or more (e.g., 1, 2, 3, 4, 5, or 6) groups independently selected from halogen, C₁₋₄ alkyl, C₂₋₄ alkenyl, C₂₋₄ alkynyl, C₁₋₄ alkoxy, C₃₋₆ cycloalkyl, and C₃₋₆ cycloalkoxy. In some embodiments, the alkyl, alkenyl, or alkynyl chain can be optionally substituted one or more (e.g., 1, 2, 3, 4, 5, or 6) groups independently selected from halogen, optionally substituted alkyl (e.g., C₁₋₆ alkyl), optionally substituted heteroalkyl (e.g., C₁₋₆ heteroalkyl, e.g., with 1 or 2 heteroatoms independently selected from oxygen and nitrogen), optionally substituted alkenyl (e.g., C₂₋₆ alkenyl), optionally substituted alkynyl (e.g., C₂₋₆ alkynyl), optionally substituted cycloalkyl (e.g., C₃₋₆ cycloalkyl), optionally substituted aryl (e.g., C₆₋₁₄ aryl), optionally substituted heterocycloalkyl (e.g., 5-8 membered heterocycloalkyl), optionally substituted heteroaryl (e.g., 5-10 membered heteroaryl), short peptides (e.g. mono, di, tri, or tetra-peptides), —NR¹⁰⁰R¹⁰¹, —C(═O)NR¹⁰⁰R¹⁰¹, —COOR¹⁰², and —OR¹⁰², wherein R¹⁰⁰, R¹⁰¹, and R¹⁰² are each independently hydrogen, optionally substituted alkyl (e.g., C₁₋₆ alkyl), optionally substituted heteroalkyl (e.g., C₁₋₆ heteroalkyl, e.g., with 1 or 2 heteroatoms independently selected from oxygen and nitrogen), optionally substituted cycloalkyl (e.g., C₃₋₆ cycloalkyl), optionally substituted aryl (e.g., C₆₋₁₄ aryl), optionally substituted heterocycloalkyl (e.g., 5-8 membered heterocycloalkyl), optionally substituted heteroaryl (e.g., 5-10 membered heteroaryl), wherein each of the optionally substituted groups is independently optionally substituted with one or more (e.g., 1-3) substituents selected from oxo, halogen, hydroxyl, NH₂, —NH(C₁₋₄ alkyl), —N(C₁₋₄ alkyl)(C₁₋₄ alkyl), C₁₋₄ alkyl optionally substituted with 1-3 fluorine, C₁₋₄ alkoxy optionally substituted with 1-3 fluorine, C₂₋₄ alkenyl, C₂₋₄ alkynyl, C₃₋₆ cycloalkyl optionally substituted with 1-3 fluorine or 1-2 C₁₋₄ alkyl, and C₃₋₆ cycloalkoxy optionally substituted with 1-3 fluorine or 1-2 C₁₋₄ alkyl. In some embodiments, In some embodiments, the short peptides can be a mono-, di-, tri-, or tetra-peptide, which can be derived from alpha-amino acids (e.g., D or L-amino acids) selected from alanine, isoleucine, leucine, methionine, valine, phenylalanine, tryptophan, tyrosine, asparagine, cysteine, glutamine, serine, threonine, aspartic acid, glutamic acid, arginine, histidine, lysine, glycine, and proline.

In some embodiments, the prodrug of morphine is characterized as having Formula 2A. In some embodiments, the prodrug of morphine is characterized as having Formula 2B. In some embodiments, the prodrug of morphine is characterized as having Formula 2C. In some embodiments, the prodrug of morphine is not a myristic ester. In some embodiments, R² in Formula 2A, 2B, or 2C is —OR²⁰ or —NHR²⁰. In some embodiments, R²′ in Formula 2B is —OR²⁰ or —NHR²⁰. In some embodiments, R²⁰ can be an unsubstituted straight alkyl chain having 7-30 (e.g., 10-24) carbons. In some embodiments, R²⁰ can be an unsubstituted branched alkyl chain having 7-30 (e.g., 10-24) carbons. In some embodiments, R² and R²′ in Formula 2 (2A, 2B, or 2C), as applicable, can each independently be an unsubstituted straight alkyl chain having 7-30 (e.g., 10-24) carbons. In some embodiments, R² and R²′ in Formula 2 (2A, 2B, or 2C), as applicable, can each independently be an unsubstituted branched alkyl chain having 7-30 (e.g., 10-24) carbons. In some embodiments, R² and R²′ in Formula 2 (2A, 2B, or 2C), as applicable, can each independently be an unsubstituted straight alkyl chain having a formula of CH₃(CH₂)ₙ—, wherein n is an integer of 8-24 (e.g., 10-24). In some specific embodiments, R² and R²′ in Formula 2 (2A, 2B, or 2C), as applicable, can each independently be selected from CH₃(CH₂)₁₀—, CH₃(CH₂)₁₂—, CH₃(CH₂)₁₄—, CH₃(CH₂)₁₆—, and CH₃(CH₂)₁₈—. R² and R²′ in Formulae 2A, 2B, and 2C are independently selected and can be the same or different from each other. R²⁰ at each occurrence is also independently selected.

In some embodiments, the present disclosure also provides specific prodrugs which can be any one of Compound Nos. 1-8 (see the Examples section), or a pharmaceutically acceptable salt thereof.

It is understood that some compounds described herein can exist as stereoisomeric forms including e.g., R—, S— and racemic (RS—) forms. Where the compound has more than one chiral centers, all diastereomers are contemplated herein. When the stereochemistry of a chiral center in a compound is specifically designated, in a drawing or otherwise, it should be understood that the compound exists mainly in the designated stereoisomeric form with regard to the chiral center, for example, with less than 20%, less than 15%, less than 10%, less than 5%, less than 1%, or non-detectable level of the other stereoisomer. Unless expressly indicated otherwise, all stereoisomer forms are contemplated herein.

The prodrugs of the present disclosure can be generally prepared by coupling the levorphanol or morphine with an agent, such as a fatty acid, fatty alcohol, or fatty amine, under suitable conditions. In some embodiments, a carbonyl donor agent, can also be used to link levorphanol or morphine with such agents. Various coupling methods can be used for the preparation of the prodrugs herein, which include those known in the art and also are exemplified in the Examples section.

For example, synthesis of the long-chain fatty acid ester prodrugs of levorphanol or morphine can be carried out using an approach shown in Example 1 and Example 2 respectively, wherein the long-chain fatty acid is converted into an activated form, such as the corresponding acyl chloride using an activating agent such as SOCl₂. The resulting activated form, such as acyl chloride, can then be coupled to the hydroxyl groups, e.g., the phenolic hydroxyl group of levorphanol or morphine, to give the long-chain fatty acid ester opioid prodrug. In view of the present disclosure, the prodrugs of the present disclosure can be readily prepared by those skilled in the art.

In another aspect, the present disclosure also provides processes and novel intermediates disclosed herein which are useful for preparing the prodrugs of the present disclosure. In other aspects, methods for synthesis, analysis, separation, isolation, purification, characterization, and testing of the prodrugs of the present disclosure are provided.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wis., USA), Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999).

### III. METHODS OF USE

The prodrugs of the present disclosure are useful for the treatment of any of the diseases or disorders where administering the parent drugs (e.g., levorphanol or morphine) are useful. For example, as levorphanol or morphine can be used as an analgesic, the prodrugs of levorphanol or morphine can also in some embodiments be used in a method for treating pain (e.g., chronic pain).

Accordingly, in some embodiments, the present disclosure provides a method of treating pain (e.g., chronic pain), the method comprising administering to a subject in need thereof a therapeutically effective amount of a prodrug of the present disclosure (e.g., Formula 1 or 2 (2A, 2B, or 2C), a long-chain fatty acid ester of levorphanol, or a long-chain fatty acid ester of morphine, or a pharmaceutically acceptable salt thereof) or a pharmaceutical composition comprising the prodrug. In some embodiments, the prodrug is a compound of Formula 1 or 2 (2A, 2B, or 2C), a long-chain fatty acid ester of levorphanol, a long-chain fatty acid ester of morphine, or a pharmaceutically acceptable salt thereof. In some embodiments, the prodrug is any one of Compound Nos. 1-8, or a pharmaceutically acceptable salt thereof. In some embodiments, the method is for treating chronic pain. In some embodiments, the method is for treating moderate to severe pain. In some embodiment, the method is for treating neuropathic pain, and the prodrug is a compound of Formula 1, a long-chain fatty acid ester of levorphanol, or a pharmaceutically acceptable salt thereof. In some embodiments, the administering can be an injection, such as a subcutaneous or intramuscular injection. In some embodiments, the administration can slowly release the corresponding parent drug (e.g., levorphanol or morphine) in vivo and provide a long-lasting therapeutic effect (i.e., long-acting effect). For example, in some embodiments, the administration provides a release of levorphanol or morphine in the subject over an extended period of time, such as over a period of at least 1 day, at least 2 days, or at least 3 days. In some embodiments, intramuscular injection prodrugs of the present disclosure can slowly release the corresponding parent drug (e.g., levorphanol or morphine) in vivo and provide a long-lasting therapeutic effect (i.e., long-acting effect). This release profile can be advantageous at least in that it allows a less frequent administration and better patient compliance. Also, as the prodrug is administered via injection, the formulation can be restricted to hospital use, which therefore can greatly reduce the likelihood of an abuser obtaining a large quantity of the prodrug for attempted extractions (or otherwise attempted tampering) of levorphanol or morphine. Further, as levorphanol or morphine or a metabolite thereof is only released slowly after administration of the prodrug of the present disclosure, the abuser's potential reward of euphoria may not be achieved by simply injecting the formulation. As such, the potential for abuse is also reduced.

In some embodiments, the prodrugs of the present disclosure can be used for preparing an abuse deterrent formulation. As described herein, the inventors also designed the prodrugs and pharmaceutical compositions comprising the same to effectively resist some or all chemical and physical conditions that are commonly used by drug abusers, including chewing, crushing, injection, and inhalation, or simple extraction with organic solvents. The term “abuse deterrent” and “abuse resistant” are used herein interchangeably, both of which do not require full prevention of abuse. Abuse deterrent or resistant properties/methods include, but are not limited to, any of the properties/methods described herein as useful for deterring drug abuse, such as properties that allow a formulation to be resistant to common hydrolysis conditions by potential abusers, methods of restricting potential abusers' access to the controlled substance, etc. The term “abuse” should be understood as the intentional, non-therapeutic use of a drug product or substance, even once, to achieve a desirable psychological or physiological effect.

In some embodiments, the present disclosure also provide methods of reducing a likelihood of abuse of levorphanol or morphine. In some embodiments, the method comprises providing a prodrug of levorphanol or morphine, and formulating the prodrug in an abuse-deterrent formulation. In some embodiments, the prodrug is a compound of Formula 1 or 2 (2A, 2B, or 2C), a long-chain fatty acid ester of levorphanol, a long-chain fatty acid ester of morphine, or a pharmaceutically acceptable salt thereof. In some embodiments, the prodrug can be a compound of Formula 1 or 2 (e.g., any of the Compound Nos. 1-8), or a pharmaceutically acceptable salt thereof. In some embodiments, the abuse-deterrent formulation is an injectable formulation, such as a subcutaneous or intramuscular injectable formulation. In some embodiments, the abuse-deterrent formulation is resistant towards (e.g., substantially stable under) common tampering conditions, such as baking soda or vinegar mediated hydrolysis at pH of about 8.3 or 2.4, respectively, or citric acid mediated hydrolysis at a pH of about 1.6. As used herein, “substantially stable” should be understood as less than 30%, less than 20%, or less than 5% degradation under a given condition, e.g., hydrolysis under common tampering conditions described herein. Thus, even if an abuser can obtain the prodrug formulation, because of the difficulty in obtaining levorphanol or morphine from the prodrug formulation, the likelihood of drug abuse is also reduced. In some embodiments, the abuse-deterrent formulation comprises micelles comprising the prodrug. Without wishing to be bound by theories, it is believed that micelle formation can further reduce the rate of hydrolysis and thus improve the formulation's stability towards common tampering conditions that attempt to recover levorphanol or morphine from the prodrug formulation. In some embodiments, the method further comprising restricting the administration of the abuse-deterrent formulation to a hospital setting. Thus, the method limits access of the prodrug formulation to a potential abuser, which also reduces the likelihood of abuse. In some embodiments, the abuse-deterrent formulation also provides a long acting release of levorphanol or morphine. For example, the abuse-deterrent formulation can, after administration, release the controlled substance, or a metabolite thereof, in a subject user over an extended period of time, such as at least 1 day, at least 2 days, or at least 3 days. As the controlled substance or a metabolite thereof is only released slowly after administration, the abuser's potential reward of euphoria may not be achieved by simply injecting the formulation. As such, the likelihood of abuse is also reduced.

Without wishing to be bound by theories, the following rationales regarding exemplary prodrugs based on long-chain fatty acids further show advantages of the prodrugs and/or methods of the present disclosure, such as the potential to reduce drug abuse by using the prodrugs of the present disclosure or pharmaceutical composition comprising the prodrugs.

Without wishing to be bound by theories, it is believed that the prodrugs based on long-chain fatty acids are stable in physical and chemical conditions to resist tampering. Ester prodrug is usually stable under normal storage conditions. In order to secure FDA approval, these marketed carboxyl ester prodrugs have to have adequate stability under normal storage conditions, usually, 18-24 months shelf life at room temperature. Carboxyl ester prodrug is also stable under common kitchen chemistry tampering conditions. The weak acidic and basic conditions generated by kitchen chemicals, such as acetic acid (pH=2.4 at 1.0M), citric acid (pH=1.57 at 1.0M) and baking soda (pH═8.3 at 1.0M), cannot hydrolyze carboxyl ester in hours even at elevated temperature.

Without wishing to be bound by theories, it is also believed that the enzyme mediated release of the controlled substance from prodrugs based on long-chain fatty acids can be adjusted. Controllable hydrolysis rate can prevent an ester prodrug from releasing the active parent drug immediately following administration into human body. Esterases lack substrate specificity in general. It is widely believed that ester prodrugs are hydrolyzed by various esterases in all tissues. However, it has been reported recently that one carboxylesterase usually predominates with each substrate and serves as the major pathway of hydrolysis.

Desirable enzyme conversion rate can be achieved by selecting an appropriate prodrug as described herein. Firstly, the enzyme catalyzed hydrolysis rate of long-chain fatty acid ester is slower than that of short-chain fatty acid ester. By selecting a longer fatty acid chain, the hydrolysis rate of the ester prodrug can be slowed. Therefore, an ‘extended’ release profile can be achieved with appropriate fatty acid chain length.

Secondly, by exploiting the difference between esterases and lipase, prodrugs based on long-chain fatty acid can be used to limit the site of enzyme conversion. Unlike most marketed short-chain fatty acid ester prodrugs, which can be hydrolyzed by either carboxylesterase or other esterases, long-chain fatty acid ester is primarily hydrolyzed by carboxyl ester lipase. The conversion rate of long-chain fatty acid ester opioid prodrug in plasma is much slower than in digestive tract. The prodrug can also form micelle that remains inactive for carboxyl esterases in plasma. The micelle is gradually taken up and cleaved by lipase in endothelial cell wall and liver, which is also a process too slow to generate the euphoria desired by drug abusers. Therefore, when inhaled or snorted, long-chain fatty acid ester opioid prodrugs exhibit an ‘extended’ release profile in the systemic circulation.

The prodrugs of the present disclosure can be formulated in an injectable formulation (e.g., intramuscular injection), which can provide a long-acting release of levorphanol or morphine over an extended period of time. As shown in PCT/US2018/042880, by changing the fatty acid chain length of representative oxymorphone or hydromorphone prodrugs, different PK profiles can be observed. Thus, different fatty acid ester prodrugs herein can be used for different applications.

The prodrugs of the present disclosure can be formulated to provide a long-acting release of levorphanol or morphine, which can reduce the likelihood of drug abuse and increase patient compliance. For example, injection (e.g., intramuscular injection) of long-chain fatty acid ester prodrugs (e.g., levorphanol or morphine palmitate or arachidate) can provide the controlled substance over a long period of time. Without wising to be bound by theories, it is believed that for moderately water-soluble compounds, the addition of long-chain fatty acid significantly decreases the solubility of prodrug. When the resulting lipophilic prodrug is injected via intramuscular route, the prodrug can form a depot at the injection site, slowly converts back to parent drug, and gradually releases into systemic circulation. This effect was observed for some of the representative oxymorphone or hydromorphone prodrugs as shown in PCT/US2018/042880.

Also without wishing to be bound by theories, it is hypothesized that lipidized compounds can achieve long-acting effects by binding to albumin or serum lipoprotein. Binding to protein stabilizes the compound and increase its circulation half-life.

In sum, the prodrugs of the present disclosure can be used to release the underlying levorphanol or morphine slowly. And when the prodrug is administered every 1-4 weeks by injection, such as intramuscular injection, which can be typically restricted to hospital use, the chances of drug abuse and diversion of levorphanol or morphine can be reduced. In addition, the consistent around-the-clock level of levorphanol or morphine in the plasma, e.g., for the treatment of chronic pain, and a decreased injection frequency can also greatly improve patient adherence and compliance. As discussed herein, the chemistry to make the prodrug is cost-effective and straightforward. And the myriad of potential fatty acids allows tailoring of prodrugs for specific properties.

### IV. PHARMACEUTICAL COMPOSITIONS

In another aspect, the present invention provides pharmaceutical compositions comprising any of the prodrugs of the present disclosure (e.g., compounds of Formula 1 or 2 (2A, 2B, or 2C), a long-chain fatty acid ester prodrug of levorphanol, a long-chain fatty acid ester prodrug of morphine, or any one of Compound Nos. 1-8, or pharmaceutically acceptable salt thereof). Typically, the pharmaceutical composition includes a pharmaceutically acceptable excipient and a prodrug of the present disclosure (e.g. Formula 1 for 2 (2A, 2B, or 2C), a long-chain fatty acid ester prodrug of levorphanol, a long-chain fatty acid ester prodrug of morphine, or any one of Compound Nos. 1-8, or pharmaceutically acceptable salt thereof), e.g., in a therapeutically effective amount. In some embodiments, the prodrug can be a compound of Formula 1 or 2 (e.g., any of the Compound Nos. 1-8), or a pharmaceutically acceptable salt thereof. In some embodiments, the pharmaceutical composition can provide a long acting release of levorphanol or morphine in a subject user. For example, in some embodiments, the pharmaceutical composition can, after administration, release levorphanol or morphine, or a metabolite thereof, in a subject user over an extended period of time, such as at least 1 day, at least 2 days, or at least 3 days.

In some embodiments, the pharmaceutical composition of the present disclosure is abuse deterrent. As explained herein, the pharmaceutical composition of the present disclosure can reduce the potential of drug abuse. For example, in some embodiments, the pharmaceutical composition can be formulated for injection, such as subcutaneous or intramuscular injection, which can be restricted to hospital use. As this reduces the availability of the formulation to a potential abuser, the likelihood of drug abuse of the controlled substance is also reduced. Further, as the pharmaceutical composition typically releases levorphanol or morphine or a metabolite thereof slowly after administration, the abuser's potential reward of euphoria may not be achieved by simply administering the pharmaceutical composition (e.g., through injection). Moreover, the pharmaceutical composition herein can also be characterized as being resistant towards (e.g., substantially stable under) common abuse conditions. For example, in some embodiments, the pharmaceutical composition is substantially stable under acid- or base-catalyzed hydrolysis conditions, e.g., with a pH of about 1-3 (acid catalyzed) or about 8-9 (base-catalyzed), such as vinegar or baking soda mediated hydrolysis, at a pH of about 2.4 or about 8.3, or a citric acid mediated hydrolysis at a pH of about 1.6. In some embodiments, the pharmaceutical composition can comprise micelles comprising the prodrug of the present disclosure. Without wishing to be bound by theories, as micelles are typically more stable towards acid- or base-catalyzed hydrolysis, the pharmaceutical composition comprising micelles of the prodrug can also be abuse deterrent.

In an exemplary embodiment, the pharmaceutical composition includes from 1 μg to 2000 mg of a prodrug disclosed herein, e.g., 1 μg to 1 mg, 1 mg to 10 mg, 1 mg to 100 mg, 1 mg to 1000mg, 1 mg to 1500 mg, or even 1 mg to 2000 mg.

The prodrugs of the present disclosure can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The prodrugs of the present disclosure can also be administered by injection, that is, intravenously, intramuscularly, intracutaneously, subcutaneously, intraduodenally, or intraperitoneally. Also, the prodrugs of the present disclosure described herein can be administered by inhalation, for example, intranasally. Additionally, the prodrugs of the present disclosure can be administered transdermally. The prodrugs of the present disclosure can also be administered by in intraocular, intravaginal, and intrarectal routes including suppositories, insufflation, powders and aerosol formulations. The pharmaceutical compositions described herein can be adapted for oral administration .

For preparing pharmaceutical compositions from the prodrugs of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of REMINGTON'S PHARMACEUTICAL SCIENCES, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the prodrug of the present disclosure mixed with a filler or binders such as lactose or starches, lubricants such as talc or magnesium stearate, and, optionally, stabilizers. In soft capsules, the prodrugs of the present disclosure may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil carriers such as a vegetable oil, such as arachis oil, olive oil, sesame oil or coconut oil, or in a mineral oil such as liquid paraffin; or a mixture of these can also be used for formulating the prodrugs of the present disclosure, for example, for an injectable formulation. In some embodiments, the oil is used as a carrier, and the prodrug is suspended in the oil carrier. In some embodiments, the oil suspensions can contain a thickening agent, such as beeswax, hard paraffin or cetyl alcohol. Sweetening agents can be added to provide a palatable oral preparation, such as glycerol, sorbitol or sucrose. These formulations can also be preserved by the addition of an antioxidant such as ascorbic acid. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The prodrugs of the present disclosure can be delivered transdermally, by a topical route, formulated as applicator sticks, solutions, suspensions, emulsions, gels, creams, ointments, pastes, jellies, paints, powders, and aerosols.

The prodrugs of the present disclosure can also be delivered as microspheres or implants for slow release in the body. For example, microspheres can be administered via intradermal injection of drug -containing microspheres, which slowly release subcutaneously; as biodegradable and injectable gel formulations; or, as microspheres for oral administration. Both transdermal and intradermal routes afford constant delivery for weeks or months.

The prodrugs of the present disclosure can also be delivered as subcutaneous (SC) or intramuscular (IM) injectable in situ depot for slow release in the body. The prodrug can be mixed with organic solvent in the syringe and remains in liquid form. After injection, the prodrug can form an in situ depot, which constantly deliver drug for weeks or months.

The prodrugs of the present disclosure can be provided as a salt which can be formed with many different types of acids, including but not limited to hydrochloric, sulfuric, acetic, lactic, tartaric, malic, succinic, etc. Salts tend to be more soluble in aqueous or other protonic solvents than the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the prodrugs of the present disclosure are useful for parenteral administration, such as intravenous (IV) administration or administration into a body cavity or lumen of an organ. The formulations for administration will commonly comprise a solution of the compound dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the prodrug in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the prodrugs of the present disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the prodrug into the target cells in vivo.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Prodrugs of the present disclosure may be metabolized by lipases or esterases. When the prodrug is metabolized by lipases or easterases, the ester bond is cleaved and the active opioid such as levorphanol or morphine is released.

Utilizing the teachings provided herein, an effective dosing regimen can be planned which can involve careful selection of active compounds by considering factors such as compound potency, relative bioavailability, release rate, patient body weight, presence and severity of adverse side effects, preferred mode of administration, and the toxicity profile of the selected agent.

### V. EXAMPLES

### Example 1—Synthesis of Levorphanol Ester Prodrugs

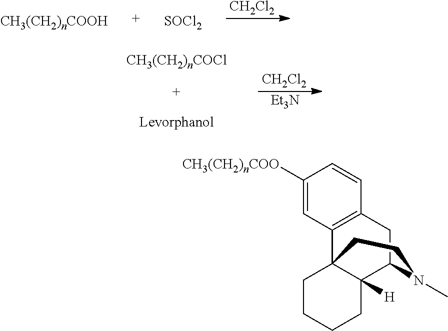

Exemplary procedure for preparing levorphanol palmitic (n=14) esters. Fatty acid (palmitic acid) and sulfonyl chloride (>10 mol eq) were added in a dry round bottom flask. The mixture was refluxed for 2 hours at 85° C. in an oil bath. The liquid sulfonyl chloride was removed under vacuum using a rotavap and further using an oil pump. Anhydrous CH₂Cl₂ was added into the mixture. The added solvent was then removed under vacuum using a rotavap and further using an oil pump. The steps of adding and removing of anhydrous CH₂CH₂ were repeated three times to ensure the residual sulfonyl chloride was removed. The obtained light yellow crystal was used for next reaction without further purification.

Levorphanol tartrate salt (1.0 mol eq) and triethylamine (4.0 mol eq) were dissolved in anhydrous CH₂Cl₂ in a round bottom flask. The flask was kept in an ice water bath. Fatty acid chloride (palmitic acid chloride) (1.1 mol eq) prepared above was added into the mixture. The mixture was stirred overnight. The mixture was washed with 0.1 N citric acid (2 times) and with water (1 time). The organic phase was collected, and dried with anhydrous K₂SO₄. The organic solvent was removed using a rotavap. The reaction mixture was then purified with silica chromatography using CH₂Cl₂: MeOH=20:1.

Following the procedure above, Compound Nos. 1-4, levorphanol esters with n=10, 14, 16 and 18, respectively, were synthesized.

Characterization of compounds No. 1-4:

Compound 1. Levorphanol dodecanate (n is 10). MS (m/z): 440.4. ¹H NMR (300 MHz, CDCl₃) δ 7.17 (1H, d), 6.97 (2H, m), 3.49 (1H, s), 2.97-3.16 (2H, m), 2.79 (3H, d), 2.3-2.7 (4H, m), 1.0-1.8 (29H, m), 0.87 (3H, t).

Compound 2. Levorphanol hexadecanate (n is 14). MS (m/z): 496.8. ¹H NMR (300 MHz, CDCl₃) δ 7.17 (1H, d), 6.95 (2H, m), 3.51 (1H, s), 2.97-3.21(2H, m), 2.80 (3H, s), 2.3-2.7 (4H, m), 1.0-1.8 (37H, m), 0.87 (3H, t).

Compound 3. Levorphanol octadecanate (n is 16). MS (m/z): 524.7. ¹H NMR (300 MHz, CDCl₃) δ 7.18 (1H, d), 6.97 (2H, m), 2.97-4.00 (3H, m), 2.79 (3H, s), 2.3-2.7 (4H, m), 1.0-1.8 (41H, m), 0.87 (3H, t).

Compound 4. Levorphanol eicosanate (n is 18). MS (m/z): 552.8. ¹H NMR (300 MHz, CDCl₃) δ 7.16 (1H, d), 6.94 (2H, m), 2.97-4.00 (3H, m), 2.71 (3H, s), 2.3-2.7 (4H, m), 1.0-1.8 (45H, m), 0.87 (3H, t).

### Example 2—Synthesis of Morphine Ester Prodrugs

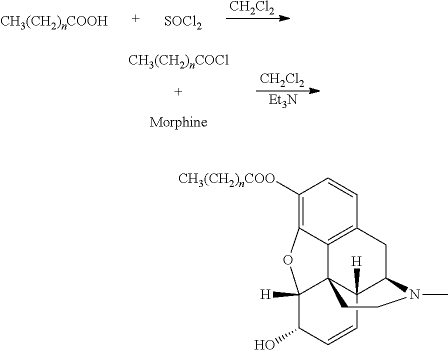

Following the procedure described in Example 1, using morphine chloride salt instead,

Compound Nos. 5-8, morphine monoesters with n=12, 14, 16, and 20, respectively, are prepared. Morphine diesters can also be prepared via similar methods, except that 2 equivalents or more of the acyl chloride are used. The other monoester can also be prepared similarly, although a protection/deprotection process may be used to enhance the reaction yield.

### Example 3—Stability Study Under Tampering Conditions

The prodrugs are subjected to common tampering condition, including 1.0M baking soda (pH=8.3), vinegar (5% acetic acid, pH=2.5), and Vodka (40% alcohol) at 80° C., and chlorine and hydrogen peroxide at 25° C. The final incubation mixture contains 10 μM test compound in a final volume of 0.5 mL tampering medium. The prodrug is added to initiate the incubation. At 0, 30, and 60 minutes, 0.05 mL aliquots is removed from the incubation mixtures and quenched with 0.15 mL of methanol and placed on ice. Aliquot is taken out for analysis. The concentration of both prodrug and parent drug is analyzed by LC-MS/MS to compare the stability of prodrugs.

### Example 4—Stability Study in Human Carboxyl Esterase and Lipase

The prodrugs are tested in recombinant human carboxyl esterase mixture containing human recombinant carboxylesterase 1b, human recombinant carboxylesterase 1c, and human recombinant carboxylesterase 2. The prodrugs are also tested in recombinant human pancreatic lipase. The hydrolysis rate in carboxyl esterase and lipase provide a ranking of the stability of the prodrugs in biological conditions.

The final incubation mixture contains 1 μM test compound and 0.1 mg/mL human recombinant carboxylesterase mixture or lipase in a final volume of 1.0 mL 0.1M potassium phosphate buffer (pH=6.0). The final percentage of DMSO in the incubation is 1.0% or less to prevent inhibition of enzymatic activity. Following a pre-incubation at 37° C., test article is added to initiate the reaction. Aliquots of 0.02 mL is removed from the incubation at 0, 30 and 60 minutes and quenched by adding 0.18 mL of freshly prepared 6N guanidinium hydrochloride solution in water containing 0.01% (v/v) phosphoric acid. The mixture is then centrifuged at 7500 g for 10 minutes at 4° C. and the supernatant is analyzed using LC-MS/MS. The percentage remaining of the prodrug and the formation of parent drug is recorded.

### Example 5—Stability Study in Human Plasma

Selected prodrugs are tested in human plasma to evaluate their stability. The final incubation mixture contains 1 μM test compound in a final volume of 1.0 mL human plasma. The final percentage of DMSO in the incubation is 1.0% or less to prevent inhibition of enzymatic activity. Following a pre-incubation at 37° C., test article is added to initiate the reaction. Aliquots of 0.02 mL is removed from the incubation at 0, 30 and 60 minutes and quenched by adding 0.18 mL of freshly prepared 6N guanidinium hydrochloride solution in water containing 0.01% (v/v) phosphoric acid. The mixture is then centrifuged at 7500 g for 10 minutes at 4° C. and the supernatant is analyzed using LC-MS/MS. The percentage remaining of the prodrug and the formation of parent drug are recorded.

### Example 6—Pharmacokinetic Study in Rats

General Procure: The formulations of opioid ester prodrugs are prepared to provide 180 mg/ml suspension by adding 1.8 g of prodrug to a 50 ml glass vial. To the solids is added 30 ml of injection vehicle. The resulting mixture is sonicated for 10 min and left standing. The contents of the vial is then shaken until a uniform, clump-free suspension is obtained prior to dosing.

Twelve male Male Sprague-Dawley rats with a body weight approximately 250 g are used in the study. A single intramuscular injection of ester prodrug (180 mg) is administered to rats. Blood samples is collected at 0 (pretreatment) 0.5, 1, 2, 4, 8, 12 and 24 days after administration. Blood is collected with commercially available plastic tubes containing a clot activator. Within 10 min of collection, blood is centrifugation at 2.500 g for 10 min. Plasma is separated and frozen at −18° C. until analyzed by LC-MS/MS. Both prodrug and its corresponding parent drug are analyzed. The pharmacokinetic parameters (AUC, Tmax, Cmax, T1/2, etc) of the test articles are compared to compare the performance of prodrugs to their parent drugs.

The formulations are prepared either in sesame oil or aqueous suspension. Both oil and suspension formulations are sterilized by electronic beam before administered to Sprague-Dawley rats.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A compound of Formula 1 or a pharmaceutically acceptable salt thereof, 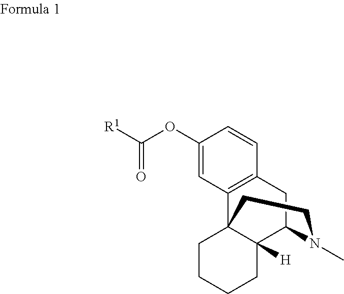
wherein R¹ is R¹⁰, —OR¹⁰, or —NHR¹⁰, wherein R¹⁰ is an optionally substituted straight or branched alkyl, alkenyl, or alkynyl chain having a total number of 7-30 carbons.

2. The compound of claim 1 or pharmaceutically acceptable salt thereof, wherein R¹⁰ is an unsubstituted straight or branched alkyl chain having 7-30 carbons.

3. The compound of claim 1, or a pharmaceutically acceptable salt thereof, wherein R¹ is an unsubstituted straight alkyl chain having a formula of CH₃(CH₂)ₙ—, wherein n is an integer of 8-24 (e.g., 10-24).

4. The compound of claim 3 or pharmaceutically acceptable salt thereof, wherein R¹ is selected from CH₃(CH₂)₁₀—, CH₃(CH₂)₁₂—, CH₃(CH₂)₁₄—, CH₃(CH₂)₁₆—, and CH₃(CH₂)₁₈—.

5. The compound of claim 1 or pharmaceutically acceptable salt thereof, wherein the alkyl, alkenyl, or alkynyl chain is optionally substituted with one or more groups independently selected from halogen, optionally substituted C₁₋₆ alkyl, optionally substituted C₁₋₆ heteroalkyl with 1 or 2 heteroatoms independently selected from oxygen and nitrogen, optionally substituted C₂₋₆ alkenyl, optionally substituted C₂₋₆ alkynyl, optionally substituted C₃₋₆ cycloalkyl, optionally substituted C₆₋₁₄ aryl, optionally substituted 5-8 membered heterocycloalkyl, optionally substituted 5-10 membered heteroaryl, short peptides, —NR¹⁰⁰R¹⁰¹, —C(═O)NR¹⁰⁰R¹⁰¹, —COOR¹⁰², and —OR¹⁰², wherein R¹⁰⁰, R¹⁰¹, and R¹⁰² are each independently hydrogen, optionally substituted C₁₋₆ alkyl, optionally substituted C₁₋₆ heteroalkyl with 1 or 2 heteroatoms independently selected from oxygen and nitrogen, optionally substituted C₃₋₆ cycloalkyl, optionally substituted C₆₋₁₄ aryl, optionally substituted 5-8 membered heterocycloalkyl, optionally substituted 5-10 membered heteroaryl,
wherein each of the optionally substituted groups is independently optionally substituted with one or more (e.g., 1-3) substituents selected from oxo, halogen, hydroxyl, NH₂, —NH(C₁₋₄ alkyl), —N(C₁₋₄ alkyl)(C₁₋₄ alkyl), C₁₋₄ alkyl optionally substituted with 1-3 fluorine, C₁₋₄ alkoxy optionally substituted with 1-3 fluorine, C₂₋₄ alkenyl, C₂₋₄ alkynyl, C₃₋₆ cycloalkyl optionally substituted with 1-3 fluorine or 1-2 C₁₋₄ alkyl, and C₃₋₆ cycloalkoxy optionally substituted with 1-3 fluorine or 1-2 C₁₋₄ alkyl,
wherein the short peptides are mono-, di-, tri-, or tetra-peptides derived from alpha-amino acids (e.g., D or L-amino acids) selected from alanine, isoleucine, leucine, methionine, valine, phenylalanine, tryptophan, tyrosine, asparagine, cysteine, glutamine, serine, threonine, aspartic acid, glutamic acid, arginine, histidine, lysine, glycine, and proline.

6. A compound of Formula 2A, 2B, or 2C, or a pharmaceutically acceptable salt thereof, 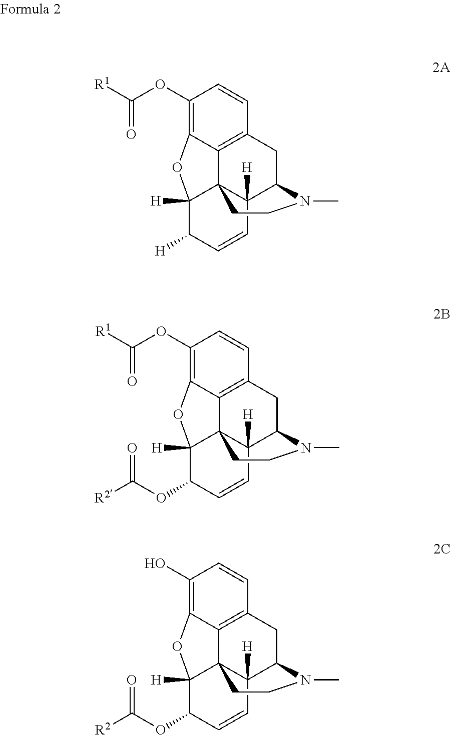
wherein R² and R²′ are independently R²⁰, —OR²⁰, or —NHR²⁰, wherein R²⁰ at each occurrence is independently an optionally substituted straight or branched alkyl, alkenyl, or alkynyl chain having a total number of 7-30 carbons.

7. The compound of claim 6 or pharmaceutically acceptable salt thereof, which has a Formula 2A.

8. The compound of claim 6 or pharmaceutically acceptable salt thereof, which has a Formula 2B.

9. The compound of claim 6 or pharmaceutically acceptable salt thereof, which has a Formula 2C.

10. The compound of claim 6 or pharmaceutically acceptable salt thereof, wherein R²⁰ is an unsubstituted straight or branched alkyl chain having 7-30 carbons.

11. The compound of claim 6, or a pharmaceutically acceptable salt thereof, wherein R² and R²′, as applicable, are each independently an unsubstituted straight alkyl chain having a formula of CH₃(CH₂)ₙ—, wherein n is an integer of 8-24 (e.g., 10-24).

12. The compound of claim 11 or pharmaceutically acceptable salt thereof, wherein R² and R²′, as applicable, are each independently selected from CH₃(CH₂)₁₀—, CH₃(CH₂)₁₂—, CH₃(CH₂)₁₄—, CH₃(CH₂)₁₆—, and CH₃(CH₂)₁₈—.

13. The compound of claim 6 or pharmaceutically acceptable salt thereof, wherein the alkyl, alkenyl, or alkynyl chain is optionally substituted with one or more groups independently selected from halogen, optionally substituted C₁₋₆ alkyl, optionally substituted C₁₋₆ heteroalkyl with 1 or 2 heteroatoms independently selected from oxygen and nitrogen, optionally substituted C₂₋₆ alkenyl, optionally substituted C₂₋₆ alkynyl, optionally substituted C₃₋₆ cycloalkyl, optionally substituted C₆₋₁₄ aryl, optionally substituted 5-8 membered heterocycloalkyl, optionally substituted 5-10 membered heteroaryl, short peptides, —NR¹⁰⁰R¹⁰¹, —C(═O)NR¹⁰⁰R¹⁰¹, —COOR¹⁰², and —OR¹⁰²,
wherein R¹⁰⁰, R¹⁰¹, and R¹⁰² are each independently hydrogen, optionally substituted C₁₋₆ alkyl, optionally substituted C₁₋₆ heteroalkyl with 1 or 2 heteroatoms independently selected from oxygen and nitrogen, optionally substituted C₃₋₆ cycloalkyl, optionally substituted C₆₋₁₄ aryl, optionally substituted 5-8 membered heterocycloalkyl, optionally substituted 5-10 membered heteroaryl,
wherein each of the optionally substituted groups is independently optionally substituted with one or more (e.g., 1-3) substituents selected from oxo, halogen, hydroxyl, NH₂, —NH(C₁₋₄ alkyl), —N(C₁₋₄ alkyl)(C₁₋₄ alkyl), C₁₋₄ alkyl optionally substituted with 1-3 fluorine, C₁₋₄ alkoxy optionally substituted with 1-3 fluorine, C₂₋₄ alkenyl, C₂₋₄ alkynyl, C₃₋₆ cycloalkyl optionally substituted with 1-3 fluorine or 1-2 C₁₋₄ alkyl, and C₃₋₆ cycloalkoxy optionally substituted with 1-3 fluorine or 1-2 C₁₋₄ alkyl,
wherein the short peptides are mono-, di-, tri-, or tetra-peptides derived from alpha-amino acids (e.g., D or L-amino acids) selected from alanine, isoleucine, leucine, methionine, valine, phenylalanine, tryptophan, tyrosine, asparagine, cysteine, glutamine, serine, threonine, aspartic acid, glutamic acid, arginine, histidine, lysine, glycine, and proline.

14. A pharmaceutical composition comprising the compound of claim 1, a long-chain fatty acid ester of levorphanol, a long-chain fatty acid ester of morphine, or a pharmaceutically acceptable salt thereof and a pharmaceutically acceptable excipient or carrier.

15. The pharmaceutical composition of claim 14, which is formulated for injection (e.g., intramuscular or subcutaneous injection).

16. The pharmaceutical composition of claim 14, which is substantially stable towards acid-catalyzed hydrolysis conditions at a pH of about 1-3 (e.g., about 1.6 or about 2.4), or base-catalyzed hydrolysis conditions at a pH of about 8-9 (e.g., about 8.3).

17. A method of treating pain in a subject in need thereof, the method comprising administering to the subject a therapeutically effective amount of the compound of claim 1, a long-chain fatty acid ester of levorphanol, a long-chain fatty acid ester of morphine, or a pharmaceutically acceptable salt thereof.

18. The method of claim 17, wherein the administration is via the subcutaneous or intramuscular route.

19. The method of claim 17, wherein the administration provides a release of levorphanol or morphine in the subject over an extended period of time, such as over a period of at least 1 day, at least 2 days, or at least 3 days.

20. A method of reducing a likelihood of abuse of levorphanol or morphine, the method comprising providing a prodrug of levorphanol or morphine, wherein the prodrug is a compound of claim 1, a long-chain fatty acid ester of levorphanol, a long-chain fatty acid ester of morphine, or a pharmaceutically acceptable salt thereof, and formulating the prodrug in a long-acting release abuse-deterrent formulation.

21. The method of claim 20, wherein the abuse-deterrent formulation is an injectable formulation, such as a subcutaneous or intramuscular injectable formulation.

22. The method of claim 20, further comprising restricting the administration of the abuse-deterrent formulation to a hospital setting, thereby limiting patient access to the compound and reducing the likelihood of abuse of the controlled substance.

23. A method of treating neuropathic pain in a subject in need thereof, the method comprising administering to the subject a therapeutically effective amount of a prodrug of levorphanol, wherein the prodrug is a compound of claim 1, a long-chain fatty acid ester of levorphanol, or a pharmaceutically acceptable salt thereof, or a pharmaceutical composition comprising the prodrug.

24. The method of claim 23, wherein the administration is via the subcutaneous or intramuscular route.

25. The method of claim 23, wherein the administration provides a release of levorphanol in the subject over an extended period of time, such as over a period of at least 1 day, at least 2 days, or at least 3 days.

26. A long-chain fatty acid ester of levorphanol, or a pharmaceutically acceptable salt thereof.
